(19) United States
(12) Plant Patent Application Publication
(10) Pub. No.: US 2019/0150337 P1
(43) Pub. Date: May 16, 2019

(54) Apple tree named `TAKANO 7GO'

(50) ;

(71) Applicant: Tsuyoshi Takano, Oshu-shi (JP)

(72) Inventor: Takao Takano, Oshu-shi (JP)

(73) Assignee: Tsuyoshi Takano, Oshu-shi (JP)

(21) Appl. No.: 15/932,601

(22) Filed: Mar. 23, 2018

(30) Foreign Application Priority Data
Nov. 11, 2017 (JP) 32562

(51) Int. Cl. A01H 6/74

(52) U.S. Cl. PLT/161

(57) ABSTRACT

A new and distinct variety of apple tree named ‘TAKANO 7GO’ having a longer common storage duration of about one month even at an average temperature of about 20-25° C., balanced sweetness and acidity of the fruit, higher firmness of the flesh, and earlier harvest time (for example, from late August to early September in Japan).

The botanical name of the novel apple tree variety is Malus Mill.

The novel variety of the Malus Mill. is hereinafter referred to by the variety denomination: ‘TAKANO 7GO’.

### CROSS-REFERENCE TO RELATED APPLICATIONS

This application claims priority to Japanese Variety Registration Application No. 32562, filed Nov. 11, 2017, which is incorporated by reference herein as if set forth in its entirety.

### BACKGROUND OF THE INVENTION

The present invention relates to a new and distinct variety of apple tree botanically classified as Malus Mill. and hereinafter referred to by the name ‘TAKANO 7GO’.

The new variety is a product of a planned breeding program conducted during a period from May 2006 to September 2017 by the inventor, Oshu-shi, Iwate, Japan, in which the Malus Mill. variety ‘Shinano Gold’ (female parent; Japanese Variety Registration No. 7328, registered Aug. 11, 1999) was crossed with the Malus Mill. variety ‘TAKANO 1GO’ (male parent) which is also known as the name ‘BENI ROMAN’ (Japanese Registered Trademark No. 5128504, registered Apr. 11, 2008) in Japan. The objective of the breeding program was to create new apple trees that produce fruits having long common storage duration and firm flesh, different from those of the parents and known similar varieties.

### SUMMARY OF THE INVENTION

The new and distinct variety ‘TAKANO 7GO’ has the following characteristics, which are distinct from: the known variety ‘Shinano Gold’ (female parent) that was produced by crossing the Malus Mill. variety ‘Golden Delicious’ which is a classic American apple variety and is also well known in Japan with the known variety ‘Senshu’ (Japanese Variety Registration No. 42, registered Mar. 31, 1980); the known Malus Mill. variety ‘TAKANO 1GO’ (male parent, Japanese Variety Registration No. 20599, registered Mar. 15, 2011) that was produced by crossing the known Malus Mill. variety ‘Shinano Red’ (Japanese Variety Registration No. 5867, registered Dec. 5, 1997) with the known Malus Mill. variety ‘Sansa’ (Japanese Variety Registration No. 1565, registered Mar. 5, 1988); and the Malus Mill. variety ‘Tsugaru,’ which is well-known in Japan.

### BRIEF DESCRIPTION OF THE DRAWINGS

The accompanying colored photographs (FIGS. 1-6) show typical flower, fruit, and leaf characteristics for the new Malus Mill. apple tree variety, ‘TAKANO 7GO.” Colors shown are as accurate as can be reasonably reproduced by photographic means. In some cases, the color might differ slightly from the colors of ‘RYOKU NH-13’ described herein.

FIG. 1 shows a tree (about 9 years old) of ‘TAKANO 7GO’ in bloom.

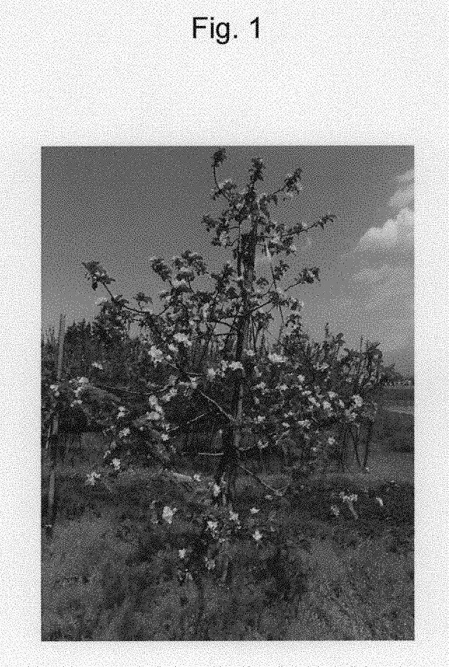

FIG. 2 shows a close-up view of the flowers of ‘TAKANO 7GO’.

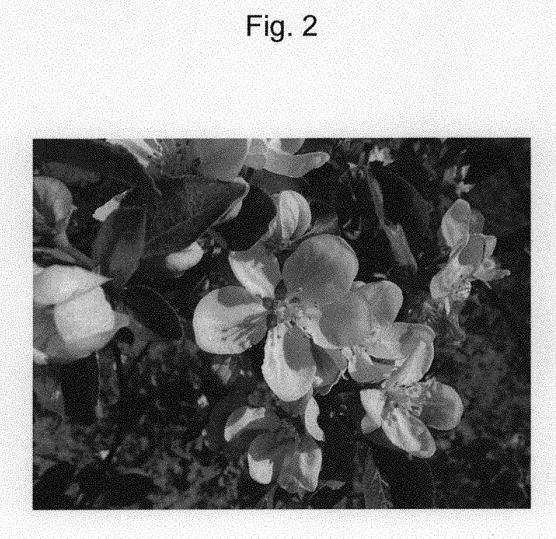

FIG. 3 shows petals, pistil, stamens and sepal of the flower of ‘TAKANO 7GO’.

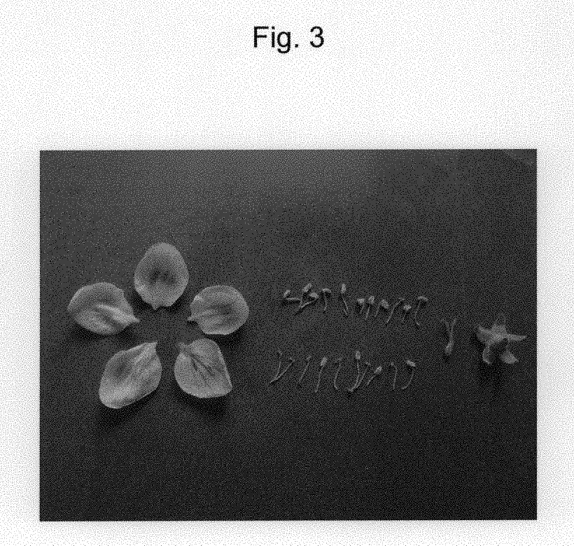

FIG. 4 shows a tree, fruits, and leaves of ‘TAKANO 7GO’.

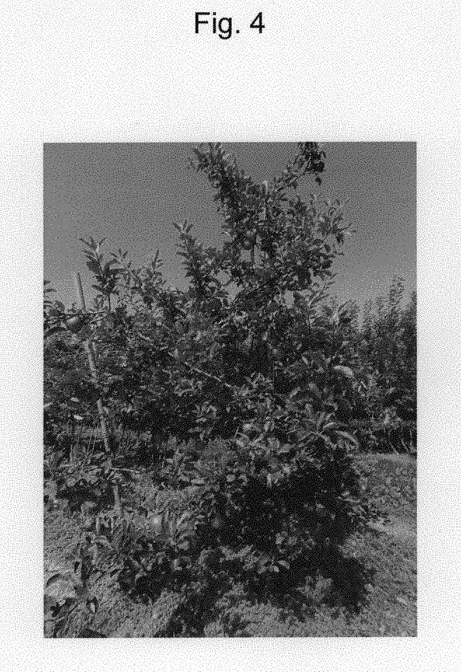

FIG. 5 shows the shape and diameter of the whole fruit of ‘TAKANO 7GO’.

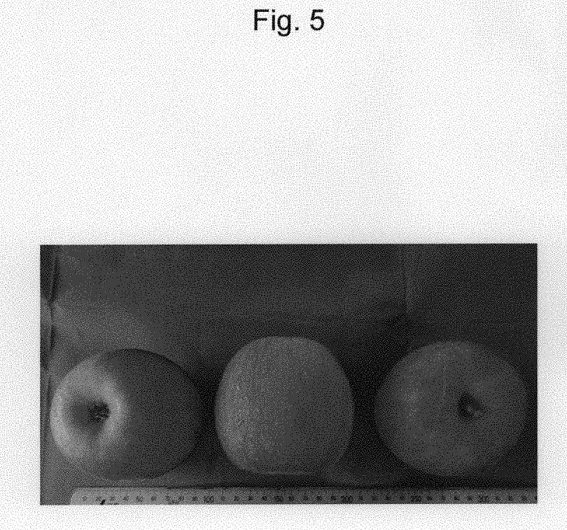

FIG. 6 shows a longitudinal cross-sectional view of the fruit of ‘TAKANO 7GO’.

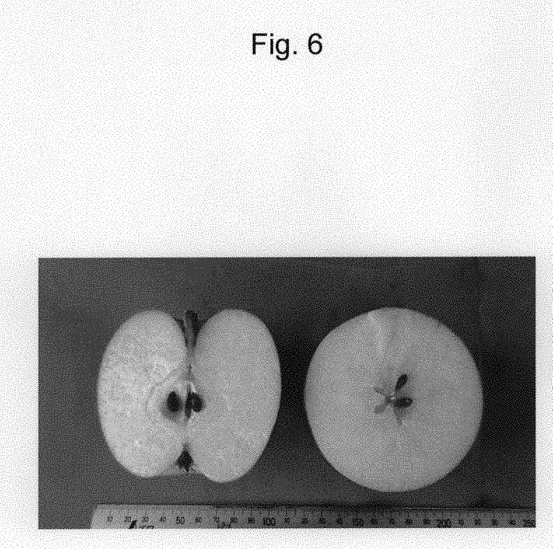

### DETAILED BOTANICAL DESCRIPTION

The new variety ‘TAKANO 7GO’ has characteristics as described below, compared with the parent varieties and similar varieties (see TABLES 1, 2 and 3).

TABLE 1
Partial characteristics of ‘TAKANO 7GO’

| Characteristic | State |
|---|---|
| Tree: type | ramified |
| Tree: habit | spreading |
| Fruit: height | medium, about 8 cm |
| Fruit: general shape | ovoid |
| Fruit: relative area of overcolor | very large |
| Fruit: hue of over color, with bloom removed | orange red |
| Fruit: intensity of stripes of overcolor | weak |
| Fruit: color of flesh | yellow white |
| Time of beginning of flowering | medium |
| Time of harvest | early |

The variety ‘Shinano Gold,’ a female parent, has the characteristics that the skin color is yellow, the sweetness of flesh is high, the acidity of flesh is medium, the food texture is firm, and the time of harvest is late, specifically late October in Iwate, Japan.

The variety ‘TAKANO 1GO,’ a male parent, has the characteristics that the skin color is red, the color of flesh is yellow white, the time of harvest is very early, specifically mid of August in Iwate, Japan, and the common storage duration is short (about 4-10 days in August in Iwate, Japan).

On the other hand, the new and distinct variety ‘TAKANO 7GO’ has the characteristics described in TABLE 1 above and TABLES 2 and 3 below, especially that the time of harvest is from late August to early September in Iwate, Japan, and the common storage duration is long (about one month even in the period from late August to early September in Iwate, Japan (average temperatures of about 20-25° C.)) substantially without making the flesh soft and, thus, very superior to any known variety produced in Japan (normally up to about 10 days). The sweetness of the fruit is about 14.5-15 and is higher than the sweetness of known fruits, such as ‘Beni Roman’ and ‘Sansa’ (about 13-14), while having balanced sweetness and acidity and higher firmness of the flesh.

TABLE 2
Comparison of characteristics between the new variety and similar varieties

| | Varieties | | |
|---|---|---|---|
| | | | Tsugaru |
| Characteristic | TAKANO 7GO | Sansa (known)^{*1)} | (known)^{*1)} |
| Stripes of over color | weak | absent | — |
| Duration of | long, | slightly short, | slightly short, |
| common storage (at | about 1 month | 10 days or less | 10 days or less |
| about 20-25° C.) | | | |
| Fruit: firmness | firm, | slightly firm, | soft, |
| of flesh | about 15.3- | about 13.8 lbs. | about 13.0 lbs. |
| | 16.0 lbs. | | |

^{*1)}‘Sansa’ and ‘Tsugaru’ are similar to ‘TAKANO 7GO’ in their harvest time.

The following detailed botanical description of ‘TAKANO 7GO’ apple tree is based on a survey (or observations) made during the period between August 2013 and September 2017 at Oshu-shi, Iwate, Japan. All colors are described according to The Royal Horticultural Society Color Chart. It should be understood that the characteristics described will vary somewhat depending upon cultural practices and climate conditions and will also vary depending upon location and season. Quantified measurements are generally expressed as an average of measurements taken from individual trees of ‘TAKANO 7GO’; however, the measurements of any individual plant or any group of plants of ‘TAKANO 7GO’ may vary from the stated average.
Classification:
   Botanical.—Malus Mill.
Parentage:
   Female parent.—Malus Mill. variety ‘Shinano Gold’ (Japanese Variety Registration No. 7328, registered Aug. 11, 1999).
   Male parent.—Malus Mill. variety ‘TAKANO 1GO’ (male parent; Japanese Variety Registration No. 20599, registered Mar. 15, 2011).
Propagation: Typically by grafting on JM1 (Japanese Variety Registration No. 7443, registered Sep. 21, 1999) rootstock; and the preferable time of grafting being the period of mid to late March in Iwate, Japan.
Flowering—beginning time: Typically in early May in Iwate, Japan.
Harvest time: Typically a period between late August and early September in Iwate, Japan.
Plant description: See TABLE 3 below indicating characteristics of ‘TAKANO 7GO’ compared with the similar variety ‘Sansa’ (Japanese Variety Registration No. 1565, registered Mar. 5, 1988).

TABLE 3
Characteristics of ‘TAKANO 7GO’ compared with ‘Sansa’

| | | | | State of | |
|---|---|---|---|---|---|
| JP Trait | UPOV | | Method of | ‘TAKANO | State of |
| No. | No. | Characteristic | Survey | 7GO’ | ‘Sansa’ |
| 1 | 1 | Tree: vigor | Observation | medium | medium |
| 2 | 2 | Tree: type | Observation (a) | ramified | ramified |
| 3 | 3 | Only barieities with | Observation (a) | spreading | spreading |
| | | ramified tree type: | | | |
| | | Tree: habit | | | |
| 4 | 4 | Tree: type of bearing | Observation (e) | on spurs and | on spurs and |
| | | | | long | long |
| 5 | 5 | One-year-old shoot: | Measurement, | medium, | medium, |
| | | thickness | mm, (b) | about 4.5 mm | about 4.5 mm |
| 6 | 6 | One-year-old shoot: length | Measurement, | short, about | medium, |
| | | of internode | cm, (b) | 2.7 cm | about 3.3 cm |
| 7 | 7 | One-year-old shoot: color | Observation (b) | medium | light brown |
| | | on sunny side | | brown | |
| 8 | 8 | One-year-old shoot: | Observation (b) | strong | medium |
| | | pubescence (on distal half | | | |
| | | of shoot) | | | |
| 9 | 9 | One-year-old shoot: number | Observation (b) | many | medium |
| | | of lenticels | | | |
| 10 | 10 | Leaf blade: attitude in | Observation (c) | upwards | upwards |
| | | relation to shoot | | | |
| 11 | 11 | Leaf blade: length | Measurement, | medium, | medium, |
| | | | cm, (c) | about 9.5 cm | about 9.8 cm |
| 12 | 12 | Leaf blade: width | Measurement, | narrow, about | narrow, about |
| | | | cm, (c) | 5.8 cm | 5.9 cm |
| 13 | 13 | Leaf blade: ratio | Measurement (c) | large, about | large, about |
| | | length/width | | 1.63 | 1.66 |
| 14 | 14 | Leaf blade: intensity of | Observation (c) | medium | medium |
| | | green color | | | |
| 15 | 15 | Leaf blade: incisions of | Observation (c) | serrate type 1 | bicrenate |
| | | margin (upper half) | | | |
| 16 | 16 | Leaf blade: pubescence on | Observation (c) | strong | medium |
| | | lower side | | | |
| 17 | 17 | Petiole: length | Measurement, | medium, | long, >2.8 cm |
| | | | cm, (c) | about 2.8 cm | |
| 18 | 18 | Petiole: extent of | Observation (c) | medium | medium |
| | | anthocyanin coloration from | | | |
| | | base | | | |
| 19 | 19 | Flower: predominant color | Obnservation (d) | dark pink | light pink |
| | | at balloon stage | | | |
| 20 | 20 | Flower: diameter with | measurement, | medium, | medium, |
| | | petals pressed into | cm, (d) | about 5.5 cm | about 5.8 cm |
| | | horizontal position | | | |
| 21 | 21 | Flower: arrangement of | Observation (d) | intermediate | intermediate |
| | | petals | | | |
| 22 | 22 | Flower: position of stigmas | Observation (d) | same level | same level |
| | | relative to anthers | | | |
| 23 | 23 | Young fruit: extent of | Observation (e) | medium | medium |
| | | anthocyanin overcolor | | | |
| 24 | 24 | Fruit: size | Observation (f) | medium | medium |
| 25 | 25 | Fruit: height | Measurement, | medium, | medium, |
| | | | cm, (f) | about 8.0 cm | about 8.0 cm |
| 26 | 26 | Fruit: diameter | Measurement, | medium, | medium, |
| | | | cm, (f) | about 8.0 cm | about 7.8 cm |
| 27 | 27 | Fruit: ratio height/diameter | Measurement (f) | very small, | small, 1.03 |
| | | | | 1.0 | |
| 28 | 28 | Fruit: general shape | Observation (f) | ovoid | conic |
| 29 | 29 | Fruit: ribbing | Observation (f) | absent or | absent or |
| | | | | weak | weak |
| 30 | 30 | Fruit: crowning at calyx end | Observation (f) | absent or | absent or |
| | | | | weak | weak |
| 31 | 31 | Fruit: size of eye | Observation (f) | small | small |
| 32 | 32 | Fruit: length of sepal | Observation | medium | medium |
| 33 | 33 | Fruit: bloom of skin | Observation (f) | absent or | absent or |
| | | | | weak | weak |
| 34 | 34 | Fruit: greasiness of skin | Observation (f) | absent or | absent or |
| | | | | weak | weak |
| 35 | | Fuit: surface texture of skin | Observation | smooth | medium |
| 36 | 35 | Fruit: ground color | Observation (f) | whitish green | whitish green |
| 37 | 36 | Fruit: relative area of | Observation (f) | very large | medium |
| | | overcolor | | | |
| 38 | 37 | Fruit: hue of overcolor-with | Observation (f) | orange red | red |
| | | bloom removed | | | |
| 39 | 38 | Fruit: intensity of overcolor | Observation (f) | medium | medium |
| 40 | 39 | Fruit: pattern of overcolor | Observation (f) | solid flush | only solid |
| | | | | with weakly | flush |
| | | | | defined | |
| | | | | stripes | |
| 41 | 40 | Fruit: width of stripes | Observation (f) | medium | narrow |
| | | | | | (absent) |
| 42 | | Fruit: intensity of overcolor | Observation (f) | weak | absent |
| 43 | 41 | Fruit: area of russet around | Observation (f) | absent or | medium |
| | | stalk attachment | | small | |
| 44 | 42 | Fruit: area of russet on | Observation (f) | absent or | medium |
| | | cheeks | | small | |
| 45 | 43 | Fruit: area of russet around | Observation (f) | absent or | large |
| | | eye basin | | small | |
| 46 | 44 | Fruit: number of lenticels | Observation (f) | many | medium |
| 47 | 45 | Fruit: size of lenticels | Observation (f) | large | medium |
| 48 | | Fruit: scarfskin | Observation | absent | absent |
| 49 | 46 | Fruit: length of stalk | Measurement, | short, about | short, about |
| | | | cm, (f) | 1.8 cm | 1.9 cm |
| 50 | 47 | Fruit: thickness of stalk | Measurement, | medium, | medium, |
| | | | mm, (f) | about 2.5 mm | about 2.4 mm |
| 51 | 48 | Fruit: depth of stalk cavity | Measurement, | medium, | medium, |
| | | | cm, (f) | about 1.3 cm | about 1.3 cm |
| 52 | 49 | Fruit: width of stalk cavity | Measurement, | medium, | medium, |
| | | | cm, (f) | about 3.7 cm | about 3.8 cm |
| 53 | 50 | Fruit: depth of eye basin | Measurement, | medium, | medium, |
| | | | cm, (f) | about 1.0 cm | about 1.0 cm |
| 54 | 51 | Fruit: width of eye basin | Measurement, | medium, | medium, |
| | | | cm, (f) | about 3.6 cm | about 3.6 cm |
| 55 | 52 | Fruit: firmness of flesh | Measurement, | firm, about | medium, |
| | | | lbs. | 15.3 lbs. | about 13.8 |
| | | | | | lbs. |
| 56 | 53 | Fruit: color of flesh | Observation (f) | yellow white | white |
| 57 | | Fruit: sweetness of flesh | Food texture | medium, | medium, |
| | | | measurement | about 14.6 | about 13-14 |
| 58 | | Fruit: acidity of flesh | Food texture | medium, | medium, |
| | | | measurement | about 0.33 | about 0.41 |
| 59 | | Fruit: water-core of flesh | Food texture | absent-very | absent-very |
| | | | measurement | slight | slight |
| 60 | | Fruit: shape of core | Measurement, | flat round, | flat round, |
| | | | cm | 2.4 × 2.4 (cm) | 2.6 × 2.6 (cm) |
| 61 | 54 | Fruit: aperture of locules (in | Observation (f) | moderately | moderately |
| | | transverse section) | | open | open |
| 62 | 55 | Time of beginning of | Observation | medium | medium |
| | | flowering | | | |
| 63 | 56 | Time of harvest | Observation | early | early |
| 64 | | Duration of storage: | Observation | long, about | slightly short, |
| | | common storage | | one month | 10 days or |
| | | | | (August, | less (August, |
| | | | | Japan) | Japan) |
| 65 | | Duration of storage: cold | Observation | long | sloghtly short |
| | | storage | | | |
| 66 | | Resistance to: Alternalia | Observation | strong | strong |
| | | blotch (Alternalia mali | | | |
| | | Roberts) | | | |

What is claimed is:

1. A new and distinct variety of Malus Mill. apple tree named ‘TAKANO 7GO,’ as illustrated and described herein.
